(19) United States
(12) Patent Application Publication
(10) Pub. No.: US 2013/0090545 A1
(43) Pub. Date: Apr. 11, 2013

(54) METHODS AND APPARATUSES FOR THE LOCALIZATION AND TREATMENT OF CANCER

(71) Applicant: Edward R. Flynn, Albuquerque, NM (US)

(72) Inventor: Edward R. Flynn, Albuquerque, NM (US)

(21) Appl. No.: 13/703,051

(22) PCT Filed: Jun. 7, 2011
(86) PCT No.: PCT/US11/39349

§ 371 Date: Dec. 10, 2012

Related U.S. Application Data
(60) Provisional application No. 61/352,782, filed on Jun. 8, 2010.

(51) Int. Cl. A61B 5/05; A61N 2/12; A61B 5/00

(52) U.S. Cl. CPC A61B 5/05; A61B 5/4836; A61N 2/12; A61B 5/4848; A61B 5/4839
USPC 600/409

(57) ABSTRACT

Embodiments of the present invention provide methods of detecting cancer, methods of treating cancer using targeted hyperthermia, methods of treating cancer using targeted chemical agents, methods of treating cancer comprising accurate measurements of the efficacy of treatments. The effect of nanoparticles on magnetic fields can be used to determine the location of a tumor, and a measure of the number of cells in the tumor. This location and measure can be used to guide therapy, and provide information regarding the most effective therapy to be applied. The same nanoparticles can be used to facilitate hyperthermia treatments, and to allow targeted application of chemical therapeutic agents.

FIELD OF THE INVENTION

This invention relates to cancer localization, treatment, monitoring of treatment and, in particular, the use of delivering hyperthermia, anti-cancer drugs, or a combination thereof, with targeted nanoparticles.

### BACKGROUND ART

Cancer is currently the second highest cause of death in the United States, second only to heart conditions. In 2009, approximately 570,000 people died of this disease, dominated by lung cancer (26-30%), breast cancer in women (15%) and prostate cancer in men (9%). Breast cancer results in 44,000 deaths a year in the United States; prostate cancer results in over 27,000 deaths. Early detection of cancer is important since discovery of tumors early minimizes the chances for metastatic transfers of the cancer to other parts of the body. Current methods for detecting cancer rely largely on (1) x-ray imaging (such as mammography in women's breast cancer), (2) ultrasound, (3) notice of physical changes, (4) MRI, and (5) PET scans. Routine screening is normally only done in mammography for women and for the genetically susceptible population. A large proportion of cancers are silent killers in that they express their presence only after metastasis and manifestation of severe discomfort. Early detection and treatment is difficult because of this and new approaches are extremely desirable.

Treatment of cancer once it is found can include any of a spectrum of methods, including surgery, chemotherapy, and ablation. Surgical removal of cancer is very effective if the cancer is contained in the primary site and is operable, but depends upon localization of the tumor. Normally, considerably more tissue is removed than the tumor itself in order to assure that all of the cancer is taken. Chemotherapy is the application of anti-cancer drugs and is normally done using injections of drugs that destroy cancer cells at a higher rate than normal cells. Typically chemotherapy is used to destroy fast growing cells and has therefore side effects affecting the digestive system and hair loss. Side effects can be severe with normal chemotherapy since the entire blood system is flooded with the chemicals. This can be particularly severe in young children with rapidly developing brains. Since traditional chemotherapy is non-localized and affects all of the body's organs it has a significant death rate associated with it. Ablation of tumors normally involves low temperature application (cryotherapy), or high temperature application (hyperthermia), to destroy cancer cells. Hyperthermia, as applied to cancer treatment, uses various devices to raise body tissue to sufficiently high temperatures, for example about 113° F. At these temperatures, cancer cells are damaged or killed. However, this must be done with minimal injury to normal tissues. The goal of cryotherapy and hyperthermia treatment is to shrink or eliminate cancerous tumors in the body. The location of the tumor must be ascertained beforehand. Typically, hyperthermia is applied over a much larger area than the known position of the tumor to make sure that the tumor is destroyed. X-rays or other imaging methods are then used to see if the tumor has been destroyed. These techniques have limitations based on sensitivity to tumor size and image contrast.

Hyperthermia is not well established as a clinical tool because studies have shown that the destruction of normal cells is excessive or there is insufficient destruction of localized tumors. A variety of methods for hyperthermia are used today including direct application of heat either localized or whole body through thermal blankets, the use of radio-frequency waves to heat up tissue, the use of optical techniques to use light (in particular infrared light), and heat probes inserted into tumors.

In general, hyperthermia is not normally used as the only therapy option when treating cancer but is combined with other forms of treatment, including radiation therapy, chemotherapy and anti-cancer drugs. Hyperthermia increases cancer cell sensitivity to other modalities, so the combination makes the other modalities more effective. When hyperthermia and radiation therapy are combined, they are often given within an hour of each other. Hyperthermia can also enhance the effects of certain anticancer drugs. The major organs exposed to hyperthermia include the breast, lungs, liver, cervix, and colon. Unfortunately, there is insufficient evidence that the current use of hyperthermia adds to patient survival times.

Hyperthermia is primarily applied locally in order to minimize damage to normal cells and organs not containing cancer. In these cases, the heating is accomplished through application of electromagnetic waves, typically in the hundreds of kHz to MHz, or the use of focused ultrasound. For implementation of these methods, the tumor position and size are predetermined by some imaging methods and the energy for heating is applied either through external means or internal probes. In this approach, the energy is focused in such a way as to minimize damage to normal tissue surrounding the tumor although inevitably damage does occur. This method is similar to cryo-ablation of tumors and removal of tumor cells, or ablation, occurs during the heating. This method is sometimes referred to as endocavitary or interstitial. Heating of larger body areas, including whole body hyperthermia, is used for larger tumors or when cancer has spread through the body. In these cases the body temperature can be raised to 108° F. either by immersion in a thermal bath or by application of RF heating over larger areas as discussed above.

In order to avoid serious side effects, the temperature of the affected regions during hyperthermia must be carefully monitored, commonly done using small inserted thermometers which are placed using various imaging devices. This can be a very painful procedure requiring local anesthesia. Normal tissue must be kept below 111° F.. Proper application of hyperthermia is related to the temperature achieved during the treatment, as well as the length of treatment and cell and tissue characteristics. Although the goal of hyperthermia is not to destroy normal tissues by keeping the tissue temperature under 111° F., differences in tissue characteristics may cause hot spots resulting in burns, blisters, discomfort, or pain. Accurate localization of where the hyperthermia is applied can reduce the destruction of normal cells and permit higher concentration of energy applied to the tumor cells.

The standard application of chemotherapy is the direct injection of drugs into the body to treat the cancer. In most applications, one or more drugs are used to treat a variety of cancers and only certain drugs are specific to distinct cancer types. The drugs can be aimed directly at cancer cells or can be aimed at the rapidly growing vascular structure associated with a tumor. Although cancer cells are most affected by these drugs, many other cell types in the body also are harmed. The side effects can be life changing. The brain can suffer severe effects, sometimes referred to as “Chemo Brain”, that can cause confusion and disorientation for years or for life. The effects on young children can cause loss of IQ and life-long memory problems. There may be short or long term skin, hair and intestinal changes. Peripheral neuropathy can also be induced and the resulting peripheral nerve damage can be permanent. Localization of drug concentration and effectiveness would allow a major change in chemotherapy application permitting the use of more advanced and specific drugs while minimizing the side effects of the drug.

In standard methods of application of therapy including hyperthermia and drug delivery, it is necessary to monitor the effects of the therapy by imaging methods that were used to determine the original location of the tumor. This is a very limiting procedure since the tumor had to have substantial size, e.g., several mm in diameter, before it could be detected originally and the knowledge of complete destruction of the tumor is limited by this detection size. Thus the normal method for monitoring the effectiveness of the treatment is to look for reoccurrence of the tumor. This also means that the treatment by hyperthermia or chemotherapy attempts to exceed the need to remove the tumor in order to make sure the therapy has succeeded resulting in increased unnecessary side effects. An effective means of monitoring the therapy would represent a major advance in the field of cancer treatment.

### DISCLOSURE OF INVENTION

Some embodiments of the present invention provide a method for using a biomagnetic imaging method to locate cancer sites at an early stage of cancer, allowing the precise determination of regions where therapy can be effectively applied. The biomagnetic method uses magnetic nanoparticles to localize the tumor by attachment to the cancer cells, as described in U.S. provisional applications 61/259,011; 61/308,897; 61/310,700; 61/329,076; 61/329,198; 61/331,816; 61/314,392; each of which is incorporated herein by reference.

In some embodiments of the present invention, the same particles can be used for hyperthermia therapy by application of external radio-frequency fields to oscillate and heat the particles and destroy the attached cells. In some embodiments, the same magnetic nanoparticles can be used to deliver anti-cancer drugs to the tumor where they are released exactly at the site of the cancer cells and not throughout the body.

Magnetic nanoparticles used in this invention are specifically targeted to the cancer cells through antibodies or angiogenesis molecules and through multi-functional coatings of the particles can carry the drugs to the specified cells. Some embodiments of the present invention provide methods to enhance drug delivery at the tumor sites by the use of external magnetic fields to concentrate the magnetic nanoparticles. The biomagnetic sensors used to locate the cancer sites can then be used to monitor the treatment to determine when the cancer cells are destroyed and the dead cells that contain attached magnetic nanoparticles have been removed by phagocytosis. The high sensitivity of the biomagnetic sensor, as used in this invention, accurately assesses the effectiveness of the treatment and minimizes the use of therapy and unnecessary side effects.

The location of tumors is obtained using magnetic relaxometry methods with sensitive magnetic systems, such as biomagnetic Superconducting Quantum Interference Detectors (SQUIDs), and injected superparamagnetic nanoparticles labeled with antibodies specific for the type of cancer being detected. Superparamagnetic nanoparticles labeled with peptides targeting the microvascular structure supplying blood to the tumor can also be suitable for localizing the tumor. The nanoparticles connect to the cancer cells in the tumor, typically several hundred thousand nanoparticles per cell. The SQUIDs are sensitive to nanogram amounts of these nanoparticles and detect tumors one thousand times smaller than conventional x-ray sensors such as mammograms.

In some embodiments of the present invention, when the precise location of the tumor is obtained, treatment can be applied by using external RF coils located exactly over the determined location of the nanoparticles as determined by magnetic relaxometry where the magnetic relaxation fields are measured by SQUID or other sensitive sensors, to oscillate the magnetic nanoparticles through their interaction with the RF field resulting in the cells, to which the nanoparticles are attached through antibodies or peptides, being heated and killed. In some embodiments of the present invention, anti-cancer drugs are attached to the magnetic nanoparticles either prior to injection or after localization of the tumor and released at the site by application of external RF heating pulse, ultrasound pulse, biochemical interaction with cell surface or other means. In some embodiments of the present invention, external magnetic fields are used to concentrate the magnetic nanoparticles containing the drugs at the tumor site. In some embodiments of the present invention, the above methods are used in combination.

Magnetic relaxometry methods using the SQUID sensors can be employed to monitor the treatment of various different modes of therapy. Measurements of the magnetic moment of the tumor due to the magnetic nanoparticles bound to the cells can be made prior to application of therapy to determine the number of cancer cells in the tumor. During the therapy, similar measurements can be performed to determine the efficacy of the treatment and to monitor cell death. Upon successful treatment, the observed magnetic moment will be reduced to the sensitivity level of the sensor system indicating all detectable cancer cells have been destroyed. The observed trend of cell destruction as a function of time of applied therapy can be used to extend the treatment beyond the detection limit to extrapolate to zero cancer cells remaining.

### BRIEF DESCRIPTION OF DRAWINGS

The accompanying drawings, which are incorporated in and form part of the specification, illustrate the present invention and, together with the description, describe the invention.

FIG. 1 is a schematic illustration of magnetic nanoparticles that can be used in the present invention for localization, hyperthermia and drug delivery in human cancer.

FIG. 2 is a graph of a measurement of the magnetic moment in a SQUID sensor system as a function of time for incubation of attaching magnetic nanoparticles to cancer cell lines in order to determine the number of nanoparticles per cell.

FIG. 3 is a photo of a mouse with tumors located by SQUID relaxometry.

Fig. 4 is an illustration of applying hyperthermia to a tumor in an animal.

FIG. 5 is an illustration of a multifunctional superparamagnetic nanoparticle containing anti-cancer drugs in a polymer coating.

### MODES FOR CARRYING OUT THE INVENTION AND INDUSTRIAL APPLICABILITY

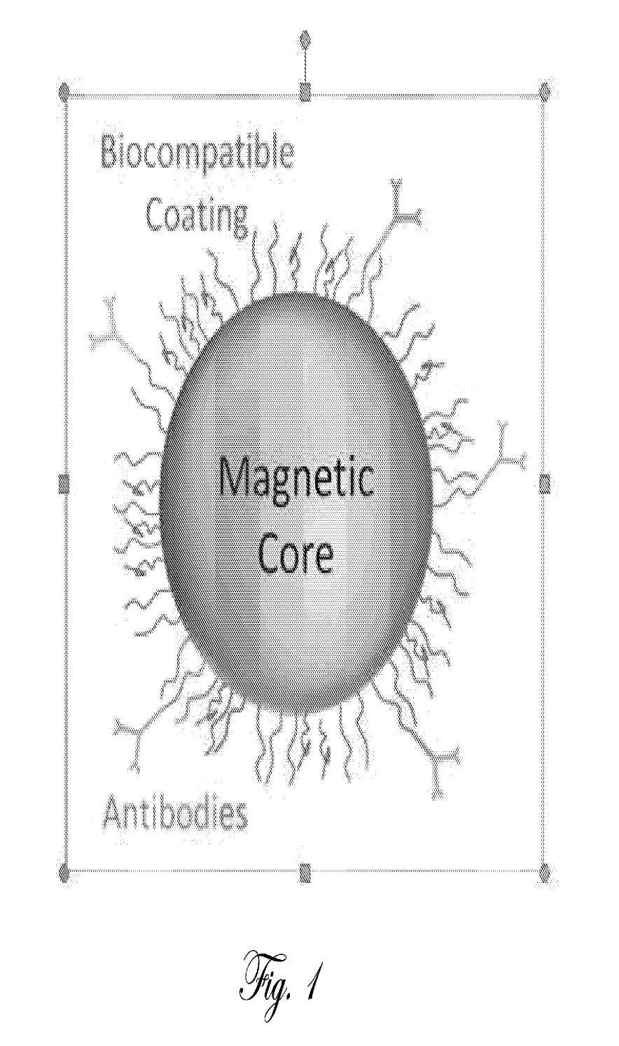

FIG. 1 is a schematic illustration of magnetic nanoparticles that can be used in the present invention for localization, hyperthermia and drug delivery in human cancer. A magnetic core comprises iron-oxide, for example magnetite or maghemite. The size of the core can be determined based on the characteristics of the magnetic system; for example a core of 24 nm in diameter can achieve desirable detection efficiency for Superconductive Quantum Interference Device (SQUID) sensors as described in the provisional applications referenced above. The superparamagnetic magnetic core is surrounded with a biocompatible surface permitting linking of various antibodies and peptides that are used for targeting the cells to be detected and for application of therapy using hyperthermia, drug delivery, or a combination of both. These antibodies link to this coating as illustrated in the figure.

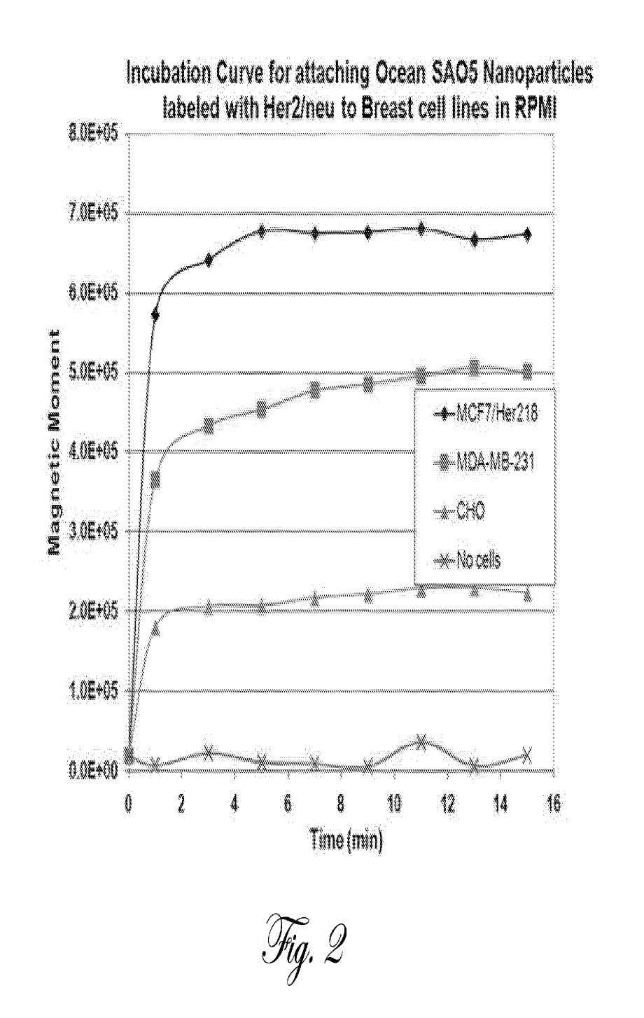

Determination of the number of nanoparticles per cell in order to determine the sensitivity for localization and the required strength of an applied field for hyperthermia can be performed by incubation measurements on cell cultures of the type of cancer under treatment. FIG. 2 is a graph of a measurement of the magnetic moment in a SQUID sensor system as a function of time for incubation of attaching magnetic nanoparticles to cancer cell lines in order to determine the number of nanoparticles per cell. FIG. 2 presents results for several breast cancer types, a non-breast cancer cell line, and for nanoparticles with no cells present. These results show that only particles attached to cell lines produce signals, and that the number of nanoparticles per cell can be used to determine the type of cancer cell present by measurement of the magnetic moment of known cells. This information can then be used to determine the sensitivity for detection and amount of hyperthermia to be applied.

For relaxometry detection and magnetic hyperthermia therapy, these nanoparticles can be injected into the blood stream or administered intratumorally. Delivery by ingestion can also be suitable in some applications. The particles are then exposed to an externally applied alternating magnetic field at the specific site identified by the SQUID sensors utilizing the relaxometry method to localize the tumor. This applied oscillating field generates heat specifically at the tumor region. The generated heat destroys cancer cells with minimal side-effects to the normal cells. The quantity of nanoparticles present in the tumor can be determined by the magnetic moment obtained from the relaxometry measurements giving the number of cancer cells present. The measurement of the number of nanoparticles connected to cells in the tumor is used to control the applied hyperthermia fields to produce sufficient heat production for cell destruction while preserving normal cells.

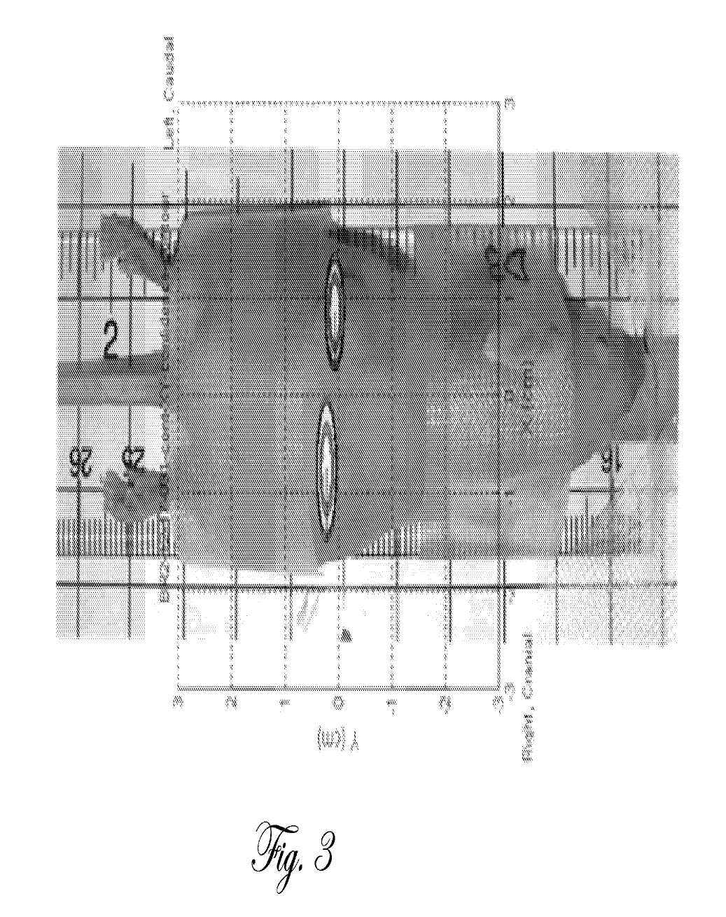

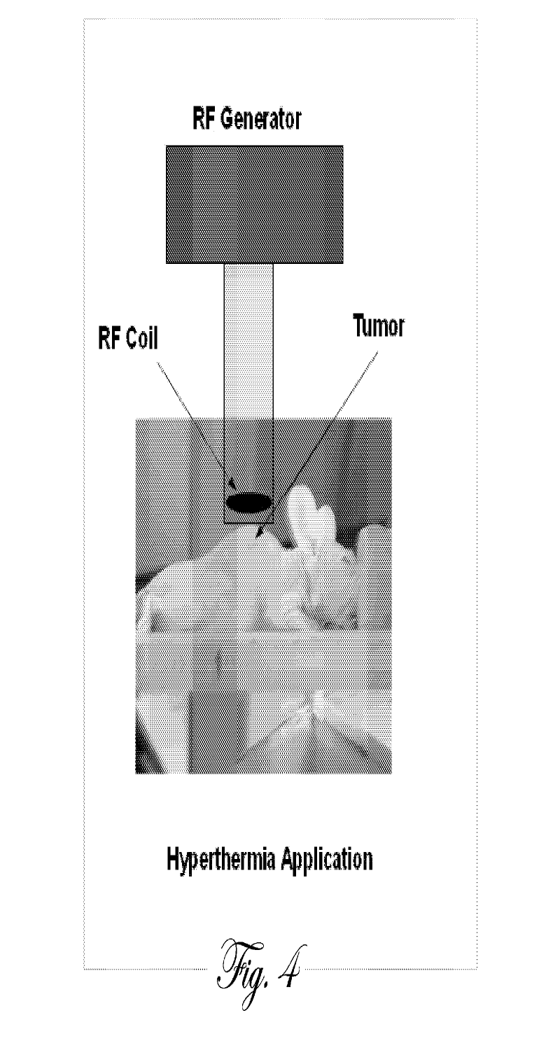

Small animal models are used to determine the capability of the SQUID relaxometry method for locating the tumors for subsequent hyperthermia therapy. FIG. 3 is a photograph of one such example. The locations of two tumors in this animal were obtained using relaxometry and superimposed on a Magnetic Resonance Image (MRI) of the animal. The ellipsoids in this figure show the confidence limits for localization as determined by magnetic relaxometry and guide the application of the magnetic field hyperthermia as illustrated in FIG. 4. The locations of the tumors by relaxometry are obtained by placement of the subject under a SQUID system and applying a short magnetization pulse to magnetize the nanoparticles that were injected to target the tumor cells. The distribution and magnitude of the resulting decaying magnetic fields is obtained by the SQUID system following the pulse. These results are compared to magnetic dipole sources through a Levenberg-Marquadt algorithm to yield the location of multiple tumors and the number of cancer cells present (through the location and total moment of the attached nanoparticles). In the example of FIG. 3 there are two tumors. This method is used to guide the hyperthermia treatment in both location and intensity and can be used as an in-vivo treatment of cancer. The localization of the application to the exact location of the tumor minimizes the side effects of the treatment, reduces the need for anesthesia, and reduces the recovery period as compared with application of hyperthermia fields over a large area of the body. Moreover, the magnetic relaxometry techniques can be used to monitor the effects of the treatment during the course of the hyperthermia application and can indicate when all of the cancer cells have been destroyed. Measurements at multiple times during treatment can be used to extrapolate the needed application of hyperthermia fields beyond the sensitivity of the SQUID system to detect the smallest tumor remaining, to determine complete destruction of the tumor.

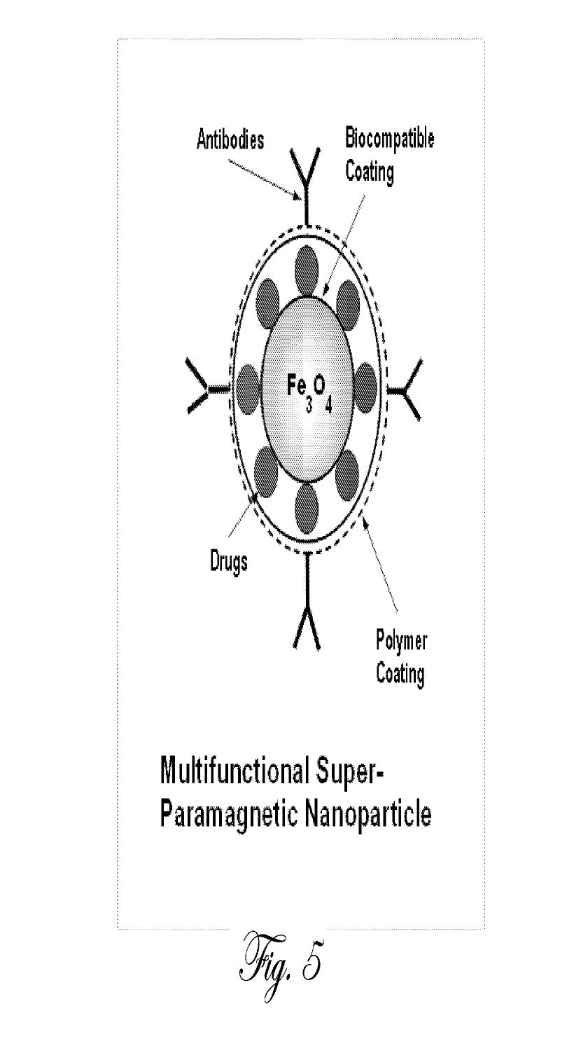

Some embodiments of the present invention use (alone or in combination with other treatments such as hyperthermia as described above) magnetic drug targeting, where multifunctional nanoparticles are used to localize and to deliver anti-cancer drugs to a tumor. The localization can be done in a similar manner as described above in relation to hyperthermia treatment. FIG. 5 is an illustration of a multifunctional magnetic nanoparticle. The core of this particle can be the same as that illustrated in FIG. 1 with the addition of drugs designed to kill the cancer cells in the tumor. Once the multifunctional nanoparticle has attached to a cancer cell, the particle can be “opened” and the drug released to kill the cancer cell. FIG. 5 illustrates one method for delivering such drugs: a polymer coating binding the drugs to the particle can be opened using an external heat pulse. Other methods known to those skilled in the art can also be used to release the drug at the site.

Multifunctional nanoparticles can be administered by intra-arterial injection or by direct injection into the tumor at a site previously identified by magnetic relaxometry. In some embodiments, the multifunctional particles can be used to both identify the location of the tumor and also to deliver the drugs to the site. After location of the tumor by magnetic relaxometry, the particles can be concentrated at the site by external magnetic field forces, for example by using strong rare-earth magnets providing high gradient fields. This method can significantly increase the amount of anti-cancer drugs at the site while minimizing the amount of these drugs in other organs such as the liver and spleen and thereby reduce side-effects normally associated with conventional anti-cancer drug chemotherapy. It is possible to increase the concentration of drugs by several orders of magnitude using magnetic concentration over normal intra-arterial injection of the drugs with no concentration. The animal model shown in FIG. 3 exemplifies this method of therapy where the external magnetic fields are applied using strong, typically conical, magnets located directly over the sites identified by the confidence limits in this photograph. Histology of the tumors from this animal model has confirmed the large presence of nanoparticles in these tumors. Significant amounts of drugs delivered are estimated by the amount of iron delivered to these tumors as determined both by the SQUID relaxometry and by histology using Prussian blue staining of the iron in the tumor slices, with several hundreds of nanograms of iron measured indicating large amounts of drugs delivered. One example method of releasing anti-cancer drugs from multifunctional particles when targeted to cells by specific antibodies or peptides is through a small amount of heat application that is done using the hyperthermia alternating magnetic field applicator. Other methods including ultrasound bursts are also effective ways to release the particles. Similar to the application of hyperthermia, the effects of this treatment can be monitored by SQUID relaxometry and the end point of treatment determined.

Numerous studies have shown that the combined use of hyperthermia and drug delivery can result in higher cancer cell death rate than the use of either individually. The use of multifunctional nanoparticles and SQUID relaxometry can be suitable for such combined use. Administration of nanoparticles to the subject can be used for localization of the tumor in using image-guided therapy. These same nanoparticles can then be used for hyperthermia and/or drug delivery through magnetic concentration as well as release of the drugs at the site. Such application of the present invention permits maximum therapy delivered at the exact site of the cancer-cell-bearing tumor while minimizing side effects due to delivery of unwanted therapy to normal cell sites, and can simultaneously provide a method for monitoring the therapy until the tumor is eliminated.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those of skill in the art.

1. A method of treating cancer, comprising (a) determining the location and number of cells in a tumor according to claim 2, and (b) applying a therapy to the patient.

2. A method for determining the location and number of cells in a tumor comprising introducing magnetic nanoparticles conjugated with biocompatible features that preferentially bind with features common to cells in the tumor, subjecting the region of the tumor to a magnetic field, measuring the effect of the nanoparticle/feature conjugates on the magnetic field, determining the number of cells in the tumor from the magnitude of the effect, and determining the location of the tumor by the location of the magnetic moment of the nanoparticle/feature conjugates bound to cancer cells.

3. A method as in claim 2, wherein the effect of the nanoparticle/feature conjugates on the magnetic field is determined by attenuating or removing the applied magnetic field and measuring the decay of the residual magnetic field.

4. A method as in claim 1, wherein step (b) comprises applying hyperthermia to the region of the tumor.

5. A method as in claim 4, wherein applying hyperthermia comprises applying an oscillating magnetic field to the region of the tumor, wherein the oscillating magnetic field generates heat by its effect on the nanoparticle/feature conjugates.

6. A method according to claim 2, further comprising (c) determining a measure of the number of cells in the tumor after treatment, then repeating steps (b) and (c) until a desired number of cancel cells have been eliminated from the tumor.

7. A method as in claim 1, wherein step (b) comprises providing delivery packages, wherein a delivery package comprises a magnetic nanoparticle, a drug that harms cancer cells, and an inhibitor that inhibits action of the drug; (b2) introducing a plurality of delivery packages into a patient; (b3) applying a magnetic field to encourage the delivery packages to the location of a tumor; (b4) treating the patient such that the inhibitor is overcome and drug acts on the cancer cells.

8. A method as in claim 7, wherein the inhibitor comprises a coating that encapsulates the drug, and wherein treating the patient so that the inhibitor is overcome comprises applying a magnetic field such that the delivery packages generate heat, motion, or a combination thereof sufficient to disrupt the inhibitory function of the coating.

9. A method as in claim 1, further comprising determining from the magnetic effect of nanoparticles in the tumor a measure of the number of cells in the tumor remaining after such treatment, (d) repeating steps (b) and (c) until the number of cells remaining in the tumor is below a threshold value.

10. A method as in claim 9, wherein the nanoparticles used in steps (a) and (c) are the same as used in step (b).

11. A method as in claim 1, further comprising (c) determining a measure of the number of cells in the tumor by the magnetic effect of nanoparticles introduced into the tumor, (d) repeating steps (b) and (c) until the number cells remaining in the tumor is below a threshold value, wherein the number of cells remaining in the tumor is determined from a plurality of measurements.

12. A method as in claim 1, further comprising (c)determining a measure of the number of cells in the tumor by the magnetic effect of nanoparticles introduced into the tumor, (d) repeating steps (b) and (c), with the treatment applied in step (b) adjusted based on the effectiveness of the treatment as evidenced at least in part from the measure of the number of cells remaining in the tumor, until the number cells remaining in the tumor is below a threshold value.

13. A method as in claim 12, wherein adjusting the treatment comprises one or more of adjusting the composition of a chemotherapy treatment, adjusting the dosage of a chemotherapy agent, changing the mode of treatment, adjusting the time of therapy application.
